(19) United States
(12) Patent Application Publication
(10) Pub. No.: US 2005/0016540 A1
(43) Pub. Date: Jan. 27, 2005

(54) Respirator product with an electric component

(76) Inventor: Werner Jumpertz, Stockelsdorf (DE)

Correspondence Address: MCGLEW & TUTTLE, PC, 1 SCARBOROUGH STATION PLAZA, SCARBOROUGH, NY, 10510-0827, US

(21) Appl. No.: 10/822,808

(22) Filed: Apr. 12, 2004

(30) Foreign Application Priority Data
Jul. 24, 2003 (DE) 103 33 585.4

(51) Int. Cl. A62B 18/00; A61M 16/00

(52) U.S. Cl. 128/205.22; 128/201.22

(57) ABSTRACT

A breathing product with an electric component has power supplied in a simple manner, without restriction of the freedom of movement of the user of the device. The breathing product has an electric connection for contacting the electric component (5) in the connection area between the breathing connection (3) and the breathing gas source (7).

### CROSS REFERENCE TO RELATED APPLICATIONS

This application claims the benefit of priority under 35 U.S.C. § 119 of German patent application DE 103 33 585.4 filed Jul. 24, 2003 the entire contents of which are incorporated herein by reference.

### FIELD OF THE INVENTION

The present invention pertains to a respirator product with an electric component.

### BACKGROUND OF THE INVENTION

Respirator products are used to supply members of task forces, for example, rescue teams, firemen and security teams, with breathing gas. Combined with a safety helmet, respirator products are frequently part of a personal protective outfit and are equipped with electric components. These components are, for example, electronic monitoring systems or an infrared camera integrated within the safety helmet.

A gas mask, in which a display unit for measured variables and status signals is arranged in the field of view of the user of the gas mask, is known from DE 36 22 147 A1. Information is displayed via the display unit on the breathing gas reserve, the ambient temperature, the humidity of the air and the concentration of harmful substances, and warning signals are generated when limit values are exceeded.

The currently available electric components are equipped with an integrated power supply. Due to this individual supply, batteries must be checked and, if necessary, replaced or charged before each mission, depending on the type of batteries used. Certain electric components, such an infrared camera integrated within the safety helmet, have limitations in terms of weight or volume, so that it is necessary to compromise in selecting the size of batteries.

It is known in the case of miner's lamps that the lamp fastened to the head can be supplied with power via a cable led separately at the head from a battery worn on the belt. Because the lamp and the battery are normally separate components, which are inserted shortly before use, the lamp and the battery can be replaced together in case of a possible damage to the cable. However, such a power supply with a separate cable is not possible in the case of electric components being worn at the head combined with display units, which are arranged within a gas mask. In case of a possible cable replacement, it would be necessary to perform repair on a plurality of individual components of the protective outfit.

### SUMMARY OF THE INVENTION

The basic object of the present invention is to improve a respirator product with an electric component such that the power supply of the electric component is made possible in a simple manner without restriction in the freedom of movement of the user of the respirator product.

According to the invention, a respirator product is provided with an electric component, a breathing connection and a breathing gas source, which can be connected to the breathing connection. An electric connecting means is provided in the connection area between the breathing connection and the breathing gas source for contacting the electric component.

The advantage of the present invention is that electric connection means, with which an electric component located at the respirator product can be contacted, are also present in the connection area between the breathing gas source and the breathing connection in addition to the gas supply. The contacting of the electric component located at the respirator product can thus also be achieved along with the establishment of the gas connection. A power supply or evaluating unit can thus be fastened in a suitable area of the user of the device, for example, on the belt. The electric connection between the evaluating unit and the electric component located at the respirator product may be established via a connecting cable, which extends in parallel to the flexible gas supply tube, or it may also be integrated within the flexible gas supply tube. The electric connecting means is advantageously designed as elastically arranged contact studs, which lie on ring-shaped strip conductors. Designing the connecting means as contactless connecting means, e.g., in the form of an inductive coupling, is also within the scope of the present invention. Such an inductive coupling has the advantage that it is not subject to any wear and cannot be damaged even during use.

The electric component is expediently designed as a measuring, monitoring, display or telemetry system. These components are either part of a gas mask, or they are integrated within a safety helmet, which is worn together with the gas mask. The electric component may also be an electrically heatable foil, which is arranged on the eye-protecting lens of a full mask and with which fogging is to be prevented.

The breathing gas source is expediently designed as a flexible breathing gas tube or as a demand oxygen system.

It is also expedient to transmit data besides an electric supply voltage via the electric connection means in the connection area between the breathing gas connection and the flexible breathing gas tube.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

FIG. 1 is a schematic view showing the design of a respirator product;

FIG. 2 is a detail view of region A according to FIG. 1 in the area of the breathing connection; and

FIG. 3 is a detail view of region A according to FIG. 1 in the area of the breathing connection in another embodiment.

### DESCRIPTION OF THE PREFERRED EMBODIMENTS

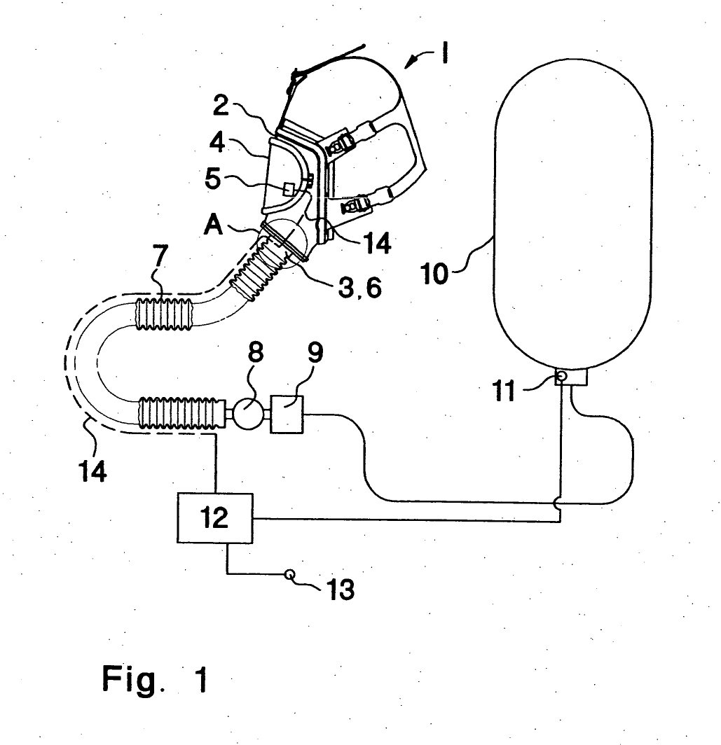

FIG. 1 schematically shows a respirator product 1 with a gas mask 2, which has a breathing connection 3 and a display unit 5 for the residual pressure, the remaining operating time and the ambient temperature. The display unit is located behind an eye-protecting lens 4. A flexible breathing gas tube 7, which is fastened with a plug 6 to the breathing connection 3, is connected to a compressed gas reservoir 10 via a gas metering unit 8 and a shut-off valve 9. A pressure sensor 11 arranged at the compressed gas reservoir 10 detects the pressure in the cylinder and is connected to an evaluating and control unit 12 with an integrated power supply. The ambient temperature is detected by a temperature sensor 13, which is likewise connected to the evaluating and control unit 12. A connecting cable 14 located at the display unit 5 extends over the breathing connection 3 and the flexible breathing gas tube 7 to the evaluating and control unit 12.

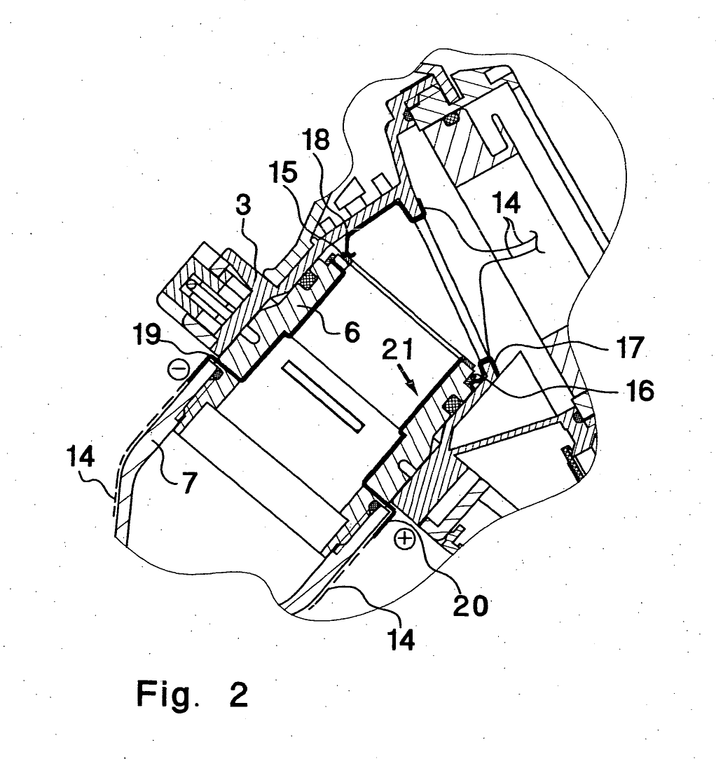

Detail A in the area of the breathing connection 3 is shown in FIG. 2 on a larger scale. Identical components are designated by the same reference numbers as in FIG. 1. Two ring-shaped strip conductors 15, 16, which are in contact with elastic contact studs 17, 18, are located on the front side of the plug 6. The contact studs 17, 18 are connected with the breathing connection 3. The connection cable 14 also connects the contact studs 17, 18 with the display unit 5. The connecting cable 14 is fastened to the outside of the flexible breathing gas tube 7 and is electrically connected to the strip conductors 15, 16 via contact strips 19, 20. The strip conductors 15, 16, combined with the contact studs 17, 18, together form a plug-type electric coupling 21 in the connection area between the breathing connection 3 and the plug 6.

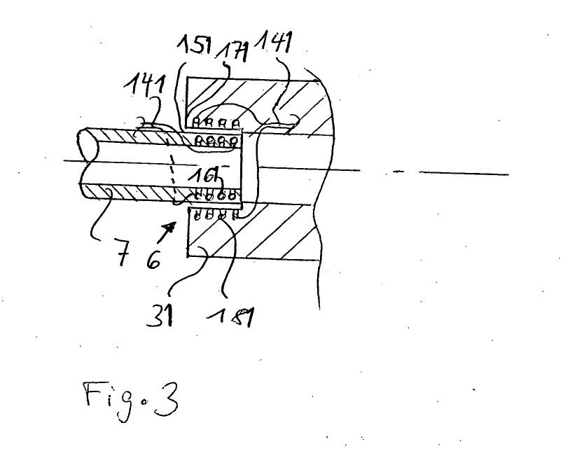

Another embodiment of detail A is shown in FIG. 3, using an inductive coupling. A plug connection 61 encompasses a plug 151 with a first inductive coil 161 and a coupling 171 with a second inductive coil 181 in the breathing connection 31. The connecting cable 141 in this embodiment is similar to connecting cable 14 in the previous embodiment on opposite sides of the inductive coupling.

The device according to the present invention operates as follows:

The pressure in the compressed gas reservoir 10 is measured continuously with the pressure sensor 11, FIG. 1. The measuring and control unit 12 calculates from this the remaining operating time that is still available to the user of the mask for the mission. The display unit 5 receives the power supply as well as the measured data for the pressure in the cylinder and the ambient temperature via the connecting cable 14. The display unit 5 is arranged in the field of view of the mask user, not shown in FIG. 1, such that both the current measured and status data and warning messages can be received, without the attention of the mask user being compromised.

The device according to the present invention is not limited to the use in a gas mask, but it is also suitable for protective clothing or for supplying pilots in the aircraft with gas.

While a specific embodiment of the invention has been shown and described in detail to illustrate the application of the principles of the invention, it will be understood that the invention may be embodied otherwise without departing from such principles.

1. A respirator product comprising:
an electric component;
a breathing connection;
a breathing gas source, which can be connected to the breathing connection; and
an electric connecting means in the connection area between the breathing connection and the breathing gas source for connection with the electric component.

2. A respirator product in accordance with claim 1, wherein the electric component is one of a measuring system, a monitoring system, a display, a telemetry system or an electrically heatable foil.

3. A respirator product in accordance with claim 1, wherein said breathing connection is arranged at a gas mask, a hood or a gas mask combined with a safety helmet.

4. A respirator product in accordance with claim 1, wherein said breathing gas source is a flexible breathing gas tube or a demand oxygen system.

5. A respirator product in accordance with claim 1, wherein the electric connection means transmits electric supply voltages and data.

6. A respirator product in accordance with claim 1, wherein the electric connection means provides a contactless connection in the form of an inductive coupling.

7. A breathing product comprising:
an electrical control;
an electric component;
a breathing connection;
a breathing gas source connected to the breathing connection; and
an electric connecting means in the connection area between the breathing connection and the breathing gas source for operative connection of said electric control with said electric component.

8. A breathing product in accordance with claim 7, wherein the electric component is one of a measuring system, a monitoring system, a display, a telemetry system or an electrically heatable foil.

9. A breathing product in accordance with claim 7, wherein said breathing connection is arranged at a gas mask, a hood or a gas mask combined with a safety helmet.

10. A breathing product in accordance with claim 7, wherein said breathing gas source is a flexible breathing gas tube or a demand oxygen system.

11. A breathing product in accordance with claim 7, wherein the electric connection means transmits electric supply voltages and data.

12. A breathing product in accordance with claim 7, wherein the electric connection means provides a contactless connection in the form of an inductive coupling.

13. A breathing product comprising:
a control unit;
an electric component; and
a releaseable inductive coupling between said control unit and said electric connection.

14. A breathing product in accordance with claim 13, further comprising:
a breathing connection;
a breathing gas source, which can be connected to the breathing connection in a connection area between the breathing connection and the breathing gas source, said releaseable inductive coupling being provided in said connection area.

15. A breathing product in accordance with claim 13, wherein the electric component is one of a measuring system, a monitoring system, a display, a telemetry system or an electrically heatable foil.

16. A breathing product in accordance with claim 13, wherein said breathing connection is arranged at a gas mask, a hood or a gas mask combined with a safety helmet.

17. A breathing product in accordance with claim 13, wherein said breathing gas source is a flexible breathing gas tube or a demand oxygen system.

18. A breathing product in accordance with claim 13, wherein the electric connection means transmits electric supply voltages and data.
